(19) United States
(12) Patent Application Publication
(10) Pub. No.: US 2024/0122622 A1
(43) Pub. Date: Apr. 18, 2024

(54) SYSTEMS AND APPARATUSES FOR NON-INVASIVE LEAD EXTRACTION TOOL WITH DUAL-ACTION ROTATIONAL AND AXIAL MOVEMENT

(71) Applicant: KONINKLIJKE PHILIPS N.V., EINDHOVEN (NL)

(72) Inventors: NATHAN C FRANCIS, COLORADO SPRINGS, CO (US); KENNETH PETER GRACE, WOODLAND PARK, CO (US)

(21) Appl. No.: 18/278,195

(22) PCT Filed: Mar. 3, 2022
(86) PCT No.: PCT/EP2022/055361

§ 371 Date: Aug. 22, 2023

Related U.S. Application Data
(60) Provisional application No. 63/159,572, filed on Mar. 11, 2021.

(51) Int. Cl. A61B 17/50

(52) U.S. Cl. CPC A61B 17/50; A61B 2017/00398

(57) ABSTRACT

Systems and apparatuses for lead extraction using a lead locking device are provided. The apparatus includes a lead extraction assistance device for use with a lead locking device (LLD) in a lead extraction procedure, the apparatus includes a lead locking device (LLD); a lead locking assist device coupled to the LLD wherein the lead locking assist device includes an assembly configured with a center shaft that has an inlet that for entry of the lead (10) and configured with an outlet for exiting the LLD wherein the assembly clamps both an entered lead and an exited LLD; and a module disposed in the assembly and coupled to both the entered lead and the exited LLD, the module configured to apply, a first action by a rotational motor (60) to twist the entering lead and a second action by a hammering device (87) for hammering the entered lead to assist in pulling out the lead.

### TECHNICAL FIELD

The technical field generally relates to endovascular procedures and more particularly relates to systems and apparatuses for enabling lead extraction by implementing a lead current lead locking device (LLD) with a device for applying vibration, hammering, and twisting motions to the lead and coupled to a proximal external portion of the lead from a body cavity to enable an unfettering of internal adhesions about the lead during the lead extraction procedure.

### BACKGROUND

The use of the lead extraction procedure is currently likely inadequate or underserved to the extent that only 30-40% of leads requiring extraction are extracted or attempted to be extracted. The reluctance by medical providers to perform lead extraction procedures is due in part to the high technique based and non-routine surgical approach required in the lead extraction, and also the small but catastrophic risk of a vascular tear in the great veins that can occur. Hence the decision to not extract the lead is weighed against the risks even when small, causing the rationale not to extract to prevail. The alleviated risk is partly because current lead extraction medical instruments require access into the great veins and a cutting of the leads away from vascular wall adhesions that have cohered to the lead. There currently is not available a medical instrument that is capable of imparting both energy and motion in a manner to the lead to have an impact or to assist in both reducing the alleviated risks caused by vascular adhesions when performing the lead extraction.

In general, a current lead extraction procedure is performed by a medical provider initially inserting a lead locking device (LLD) down the lead lumen and pulling on the lead statically to determine if the lead is removable. If the lead is deemed immovable, then the medical provider is provided with the single option of having to insert via the lead lumen, a cutting-based instrument to assist in extracting the lead and overcoming the fixation caused in part by the vascular adhesions to the lead. It is estimated that approximately 70-80% of lead extraction cases utilize this singular available alternative of inserting the cutting-based extraction instrument (i.e., laser or mechanical cutting sheath) to perform the lead extraction.

However, the use of current medical instruments that utilize cutting mechanisms presents certain drawbacks that when implemented intravascularly that include complications resulting from slicing the lead with vessel adhesions affixed to a vessel wall that is only 1 mm thick. Given the limited width of approximately 1 mm of the vessel wall and the high risk of complications, only a limited number of medical providers have sufficient experience performing and are experienced in performing the cutting procedure and have performed the cutting extraction procedure. Hence, it is commonplace for medical providers to avoid the complication risk and to prefer to cap a lead and leave the lead permanently in the body, increasing the chance for infection.

It is desirable to implement a lead locking device implement that provides no cutting mechanism inside the body to alleviate the risk of complications in the lead extraction procedure.

It is desirable to implement a lead locking device configured with a device for applying vibrating, hammering, and twisting motions to the lead which is coupled to the external portion of the lead from the body cavity for enabling a non-invasive extraction medical instrument where energy and motion are applied to the lead externally to the body cavity and that enables the unfettering of internal adhesions about the lead during a lead extraction procedure.

Accordingly, technologically improved systems and methods for valve resection and reshaping using a catheter with a resection tool to perform the is desirable. The following disclosure provides these technological enhancements, in addition to addressing related issues.

### BRIEF SUMMARY

This summary is provided to describe select concepts in a simplified form that are further described in the Detailed Description. This summary is not intended to identify key or essential features of the claimed subject matter, nor is it intended to be used as an aid in determining the scope of the claimed subject matter.

In one exemplary embodiment, a lead extraction assistance device for use with a lead locking device (LLD) in a lead extraction procedure is provided. The apparatus includes a lead locking device (LLD); a lead locking assist device coupled to the LLD wherein the lead locking assist device includes: an assembly configured with a center shaft that has an inlet for entry of the lead and configured with an outlet for exit of the LLD wherein the assembly clamps both an entered lead and exited LLD; and a module disposed in the assembly and coupled to both an entered lead and an exited LLD, the module configured to apply about the center shaft the first action by a rotational motor to twist the entered lead and a second action by a hammering device to impact the entered lead to assist to draw out the lead and counter any resistance caused by affixed vascular adhesions during the lead extraction procedure.

In at least one exemplary embodiment, the apparatus includes a non-invasive device that is exterior to a body cavity in the lead extraction procedure; and wherein the hammering device includes a hammer drill.

In at least one exemplary embodiment, the apparatus includes a spur gear which is fixed to the center shaft, wherein the center shaft is coupled on one end to the entered lead and the other end to the exited LLD, and mates to another gear of the rotational motor to translate rotational motion to the center shaft and the entered lead for application of the twist-action to twist the lead to assist in the attempt to draw out the lead and counter any resistance caused by the affixed vascular adhesions during the lead extraction procedure.

In at least one exemplary embodiment, the apparatus includes a housing of the lead extraction assistance device configured for ergonomic use in the application of a pulling action after locking of the LLD to the lead, to draw out the lead in combination with the application of the twist-action and the impact action to draw out the lead and counter any resistance caused by affixed vascular adhesions during the lead extraction procedure.

In at least one exemplary embodiment, the apparatus includes a set of pins coupled to a vane assembly with a vane collar that is attached to the assembly that is responsive to rotations of the center shaft by the rotational motor that causes the set of pins to rotate along a vane shaft of the vane assembly that results in a push action to push the vane shaft in an opposite direction and to compress a spring attached to the assembly until the set of pins reach an end of the vane shaft resultant in a release action of the compressed spring causing the hammering action that impacts the lead to draw out the lead and counter any resistance caused by affixed vascular adhesions during the lead extraction procedure.

In at least one exemplary embodiment, the apparatus includes an alternate hammering device configured with a collar and shaft wherein the collar and shaft each have opposing sawtooth designs that rotate to mimic the hammering action that impacts the lead to assist to draw out the lead and counter any resistance caused by affixed vascular adhesions during the lead extraction procedure.

Furthermore, other desirable features and characteristics of the system and method will become apparent from the subsequent detailed description and the appended claims, taken in conjunction with the accompanying drawings and the preceding background.

### BRIEF DESCRIPTION OF THE DRAWINGS

The present application will hereinafter be described in conjunction with the following drawing figures, wherein like numerals denote like elements, and:

FIG. 1 illustrates an exemplary diagram of a structure of the non-invasive tool for lead extraction implementing a hammering device and torque device, in which the extraction tool is not required to penetrate the body cavity during the lead extraction procedure to assist in the lead extraction procedure in accordance with an exemplary embodiment;

FIG. 2 illustrates an exemplary diagram of an alternative exemplary embodiment of replacing the hammering with a vibration source of the lead extraction assistance device in accordance with an embodiment;

FIGS. 3A and 3B illustrate exemplary diagrams of alternative configurations of the hammering device that enable similar impact actions as caused by hammerings illustrated in FIG. 1, of the lead extraction assistance device in accordance with an embodiment; and

FIG. 4 illustrates is an exemplary diagram of implementing a combination of the hammering device and a vibration source described in FIGS. 1 and 2 in a single assembly in the lead extraction assistance device in accordance with an embodiment.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Thus, any embodiment described herein as “exemplary” is not necessarily to be construed as preferred or advantageous over other embodiments. The embodiments described herein are exemplary embodiments provided to enable persons skilled in the art to make or use the invention and not to limit the scope of the invention that is defined by the claims. Furthermore, there is no intention to be bound by any expressed or implied theory presented in the preceding technical field, background, summary, or the following detailed description.

With increased numbers of cardiac implantable electronic devices (CIEDs) such as pacemakers, defibrillators (ICD), and cardiac resynchronization therapy (CRT) devices in patients, and consistent with the higher numbers of CIED implanted, there is also more complications, infections, and malfunctions by the greater number of CIEDs in use that necessitate more lead extraction procedures. A rationale for lead removal is given for both infectious and non-infectious conditions: the non-infectious condition can include malfunctioning leads or leads which can cause harm to the patient while the presence of an infection is for infection-based lead removal.

Exemplary embodiments provide a technical solution to this problem in the form of a lead extraction deployment mechanism that enables deploying a pacemaker/defibrillator lead extraction tool that frees up lead from adhesions without placing a cutting tool inside the body. The lead extraction tool implements a combination of vibration, hammer-drilling or succussive thrust actions, and twisting motions on the external portion of the lead to free or relieve the lead from internal affixed adhesions. The lead extraction tool is configured to lower the risk of complication by enabling use while not having to be placed within any portion of the body cavity during the procedure thereby designating the tool and a non-invasive medical instrument. Because the tool is non-invasive, there is an increased willingness for medical providers to use or to try to use the tool in the lead extraction procedure as the associated complication risks are deemed low. By overcoming the obstacle of and appearance of heightened medical risks, it is believed there will be faster and greater adherence by medical providers with acceptance combined with medical training, and prevalent use of the tool in lead extraction procedures.

Provided embodiments in the present disclosure describe systems and apparatuses of an LLD that include an extraction tool that clamps to the lead and LLD in two separate locations. When a vibration or hammering force is applied from a switched DC source coupled to the extraction tool, the vibration, hammerings, or twisting forces are received by the LLD and it causes motion about the lead in the vessel to split, fracture, or break the vascular adhesions by lateral and longitudinal as well as circular forces to enable the medical provider via manual manipulation to extract the lead. Alternately, the medical provider who is unable to extract the lead can abort the procedure and can either extract or not extract the LLD after it has been deployed.

In an exemplary embodiment, the present disclosure describes systems and apparatuses that enable Within the extraction tool there is a mechanism that twists the lead/LLD to break the mechanical adhesion between fibrous tissue and lead, along with a mechanism to apply mechanical vibration to the lead/LLD.

In an exemplary embodiment, the present disclosure describes systems and apparatuses that enable an external section configured with a voltage source wherein the vibration generates micromovements of the lead to work itself out of vascular adhesions. The housing of the extraction tool is designed to have the medical provider (i.e., the physician) hold it and apply tension to the lead, then activating the rotation and vibration, while pulling the lead out of the patient That is the housing is also configured with a convenient design for the medical provider (i.e., a physician performing the endoscopic procedure) to hold and to apply with manual traction, and subsequent lead extraction.

The figures and descriptions below provide more detail.

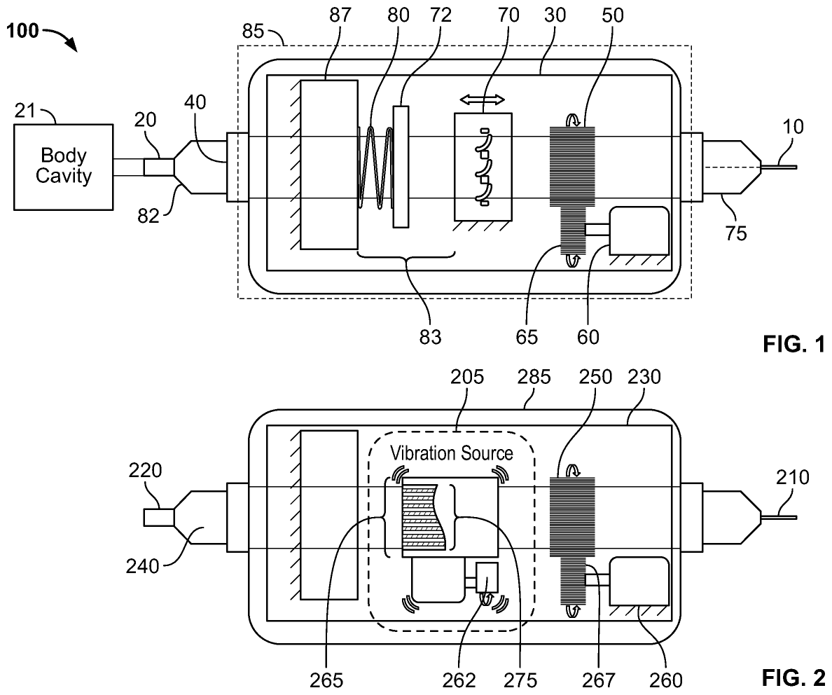

Turning now to FIG. 1 in an embodiment, FIG. 1 illustrates an exemplary diagram of a structure of the non-invasive tool for lead extraction implementing a hammering device like a hammer drill and torque device, in which the extraction tool is not required to penetrate the body cavity during the lead extraction procedure to assist in the lead extraction procedure. The lead extraction assistance device 100 requires a lead locking device (LLD) 10 for coupling to or clamping around a center shaft 40 of the assembly 30 of lead extraction assistance device 100 to transfer the various types of forces to the lead 20 that include vibration, hammering forces, and rotational forces down to the lead tip or distal end.

In the exemplary embodiment, the LLD 10 used can be either an off-the-shelf LLD or specially configured LLD with enhanced features for the response to propagated vibration, hammering, and rotation motion applied by the lead extraction assistance device 100. For example, the SPECTRANETICS® Lead Locking Device (LLD) is an off-the-shelf LLD that may be utilized with the lead extraction assistance device 100 for transvenous removal of implanted pacing or defibrillator leads.

In an exemplary embodiment, the LLD 10 is inserted by the medical provider and locked into the lead 20 that is exterior to the body cavity 21. Hence, both the lead 20 and the LLD 10 are clamp at approximately the center shaft 40 (i.e., mandrel) of the assembly 30 of the lead extraction assistance device 100. The lead 10 is inserted into the lead extraction assistance device 100 on an (exterior) exited or exiting side 75 (or proximal end) through the center shaft 40 while the lead 20 is inserted on an interior side (or distal end) 80 of the lead extraction assistance device 100. The assembly 30 of the lead extraction assistance device 100 clamps both the lead 20 and LLD 10 at either end.

That is, assembly 30 is connected to an entering (i.e., ingress) lead (i.e., at ingress or entering side 82) and the exited (i.e., exited) LLD (i.e., at an exited or exit side 75). The center shaft 40 applies a torque action by the rotational motor 60 to twist the entering (ingress) lead. The hammering device (i.e., hammer drill) 87 in the assembly 30 applies an impact or hammering actions to the entered lead on the entered side 82 to help the medical provider to manually work, to pull, or draw out the lead 20 that is resistive to the pulling action by the medical provider because of affixed vascular adhesions to the lead 20 connected with the vascular walls in the artery.

Fixed on the center shaft 40 is a spur gear 50 that mates up to a thinner spur gear 62 on a rotational motor 60 to translate rotational motion (or rotational) to the shaft/lead/LLD and twist the lead 20 to split or break the vascular adhesions. Also affixed to the center shaft 40 is a pin and vane assembly 70, with the vane collar 72 attached to a housing of the assembly 30. As the rotational motor 60 applies a rotational to turn the center shaft 40, the pins rotate along the vane path, pushing the shaft to the left, overcoming, or countering the force or energy of a spring 80 affixed to the vane collar 72. At the end of the vane path, the shaft pin slams back (resets) to the right, creating the hammer-drill effects (of instantaneous and successive impacts, or hammering type force) on both the lead 20 and the LLD 10.

In an exemplary embodiment, the pins are connected to a vane assembly with a vane collar that is attached to the assembly 30 and rotates as torques are applied to center shaft 40 by the rotational motor 60. This causes the pins to rotate down or along the vane shaft of the vane assembly and to exhibit a push action to push the vane collar 72 down the shaft in an opposite direction and to compress a spring 80 attached to vane collar 72 until the set of pins reach an end of the vane shaft 83. At this point, the spring 80 is released from a compressed state to a non-compressed state causing the series of hammering actions (i.e., mimic a push back and forth) that impacts the lead to draw out the lead and counter the resistance of lead 20 to being pulled out by the adhesions in the artery affixed to the lead 20. That is, the back and forth action break the adhesions so that the medical provider can pull out the lead 20 by gripping the ergonomic housing 85 and exerting a manual pulling force.

In an exemplary embodiment both the rotational movement to twist the lead 20 from adhesions and the hammering (hammer-drill) movement to pull the lead 20 out of the adhesions is generated by the same rotational motor 60. That is, the rotational motor 60 applies a torque to the center shaft 40 and a translational longitudinal force to the center shaft 40 that results in both types of forces transmitted to the lead 20 and the LLD 10 that are connected on either end of the center shaft 40.

The housing 85 covering the assembly 30 is configured for ergonomic use, for the medical provider to grip or hold to exact a pulling action after locking of the LLD 10 to lead 20, to draw out the lead 10 in combination with the application of the twist-action transmitted to the lead 20 and the impact action transmitted to the lead 20 to draw out the lead and counter any resistance caused by the vascular adhesions affixed to the lead 10 in the body cavity 21.

FIG. 2 illustrates an exemplary diagram of an alternative exemplary embodiment of replacing the hammering device (hammer drill) with a vibration source of the lead extraction assistance device in accordance with an embodiment. In alternative embodiments of the lead extraction assistance device, the vane/pin module may be replaced by a vibration source 205, which is composed of either an eccentric rotating mass (ERM) motor, a linear resonant actuator (LRA), or a piezoelectric actuator. The vibration source 205 would be attached to the center shaft 240 via collar 265 and needle bearing 275 to allow center shaft 240 to rotate while keeping the vibration source static, but still transmitting vibration to center shaft 240, lead 220, and the LLD 210. Any source of vibration or mechanical movement could be implemented in this design. The tool housing 285 would be ergonomic to allow the physician to grasp the device and apply traction to lead 220 and the LLD 210.

In an exemplary embodiment, the tool housing 285 is configured for ergonomic use in the application of a pulling action after locking of the LLD 210 to the lead 220, to draw out the lead 220 in combination with the application of the twist-action by the rotational motor 260 and the vibration action by the vibration source 205 to draw out the lead 220 and counter any resistance caused by affixed vascular adhesions during the lead extraction procedure.

The spur gear 250 is fixed to the center shaft 240 and the center shaft 240 is connected on one end to the entered lead (i.e. the lead 220) and the other end to the exited LLD (i.e. the LLD 210) of the assembly 230, and mates to another gear 267 of the rotational motor 260 to translate rotational motion to the center shaft 240 and to the entered lead for application of the twist-action to twist the lead 220 to assist in the attempt to draw out the lead 220 and counter any resistance caused by the affixed vascular adhesions during the lead extraction procedure. The twist-action is also applied to the LLD 210 as via the center shaft 240 coupled to the spur gear 250 and the rotational motor 260. In an exemplary embodiment, the vibration source 205 is coupled to the center shaft 240 via a collar 265 and a needle bearing 275 to enable the center shaft 240 to rotate whilst the vibration source 205 is kept static.

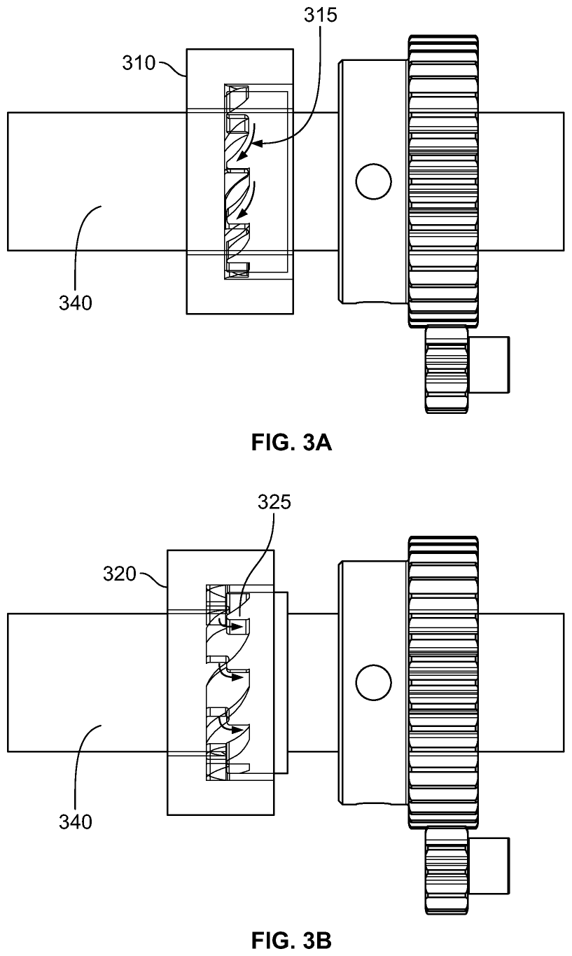

FIGS. 3A and 3B illustrate exemplary diagrams of alternative configurations of the hammering device or hammer drill that enable similar impact actions as caused by the series of thrusts or hammer-drill motion illustrated in FIG. 1, of the lead extraction assistance device in accordance with an embodiment. In FIG. 3A, in the sawtooth collar design 310, there is shown a set of teeth configured in a first direction 315 and FIG. 3B there is shown an opposing sawtooth collar design 320 with an opposing set of teeth configured in a second direction 325 that opposed the first direction. That is the movement caused by the center shaft 340 rotates in opposite direction because of the opposing sawtooth collar designs in each of the FIGS. 3A, 3B). The opposing sawtooth design collars (310, 320) and center shaft 340 forces the center shaft 340 back and forth during a rotation.

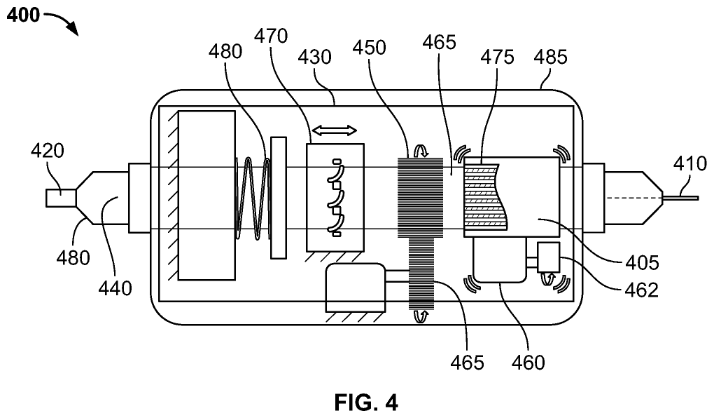

FIG. 4 illustrates is an exemplary diagram of implementing a combination of the hammering device or hammer drill and a vibration source described in FIGS. 1 and 2 a single assembly in the lead extraction assistance device in accordance with an embodiment. In FIG. 4, the combination lead extraction assistance device 400 includes the assembly 430 that clamps the entered lead 420 and the exited LLD 410. The housing 485 is configured with the ergonomic grip for the medical provider to exert a pulling action on the lead 420. Assembly 430 includes the various components described in FIGS. 1 and 2, of a spring 480, the vane assembly 470, the spur gear 450, the needle bearing 475 to the vibration source 405, the rotational motor 460, the center shaft 440. Also shown is another gear 465 connected to the spur gear 450 to complete the connection to the rotational motor 460. The combination lead extraction assistance device 400 is configured with the series/succession of hammer actions and the vibration action to apply to the center shaft 440, a torque by the rotational motor 460 to twist the lead 420 and LLD 410, to apply a hammering to impact the clamped lead 420 via the center shaft 440, and a vibration source 405 to apply a continuous or intermittent vibration to the clamped lead 420.

In an exemplary embodiment, the vibration action can be applied whilst the hammering action is applied and it can be applied in either a continuous manner or non-continuous manner to assist the medical provider to pull the lead 420 while holding or gripping the ergonomic housing 485 of the lead extraction assistance device. In another alternate exemplary embodiment, likewise, the vibration action can be applied whilst the twisting action is applied to the lead 420 and again the vibrations can be applied continuously or intermittently as the medical provider works to pull out the lead 420 from the artery. The combination lead extraction assistance device 400 provides a convenient means to switch back and forth between combinations of multiple actions of vibration, hammering, and twisting during the lead extraction procedure by using a single device to counter the resistance that can be experienced by the vascular adhesion. For example, the medical provider can apply multiple different types of forces to break the adhesions and extract the lead 420. Further, the medical provider can select a particular action that proves better in countering the resistance caused by the vascular adhesions. Also, the medical provider can choose to rotate between the set of actions to apply different forces that may have different effects to weaken the adhesions to the lead 420.

In various exemplary embodiments, the apparatus includes a lead extraction assistance device for use with a lead locking device (LLD) in a lead extraction procedure that includes the lead locking device (LLD). The lead locking assist device is coupled to the LLD and the lead locking assist device includes an assembly configured with a center shaft that has an inlet for entry the lead and configured with an outlet for exiting the LLD. The assembly clamps both an entered lead and an exited LLD. A module is disposed in the assembly and connect to both the entered lead and the exited LLD. The module is configured to apply about the center shaft the first action of a continuous, non-continuous, intermittent, or a desired length vibration to the clamped exiting lead. A second action is also applied of a twisting to the clamped entered lead whilst the continuous vibration is applied (or non-continuous vibration etc.) to assist in attempting a drawing out of the lead that is resistant to extraction because of the affixed vascular adhesions.

In another exemplary embodiment, an apparatus includes a lead extraction assistance device for use with a lead locking device (LLD) in a lead extraction procedure. The apparatus includes a lead locking device (LLD), and the lead locking assist device is connected to the LLD. The lead locking device includes an assembly configured with a center shaft that has an inlet for entry of the lead and configured with an outlet for the exit of the LLD. The assembly clamps both an entered lead and an exited LLD. A module is disposed in the assembly and coupled to both the entered lead and the exited LLD. The module is configured to apply about the center shaft the first action by a rotational motor to twist at least the clamped entered lead, a second action by a hammering device to impact the clamped entered lead, and a third action by a vibration source to apply a continuous vibration to the clamped entered lead wherein the third action of the continuous vibration is applied during application of either the first action by the rotational motor of twisting the clamped entered lead or the second action by the hammering device of impacting with hammering actions, the clamped entered lead to assist to draw out the lead and counter any resistance caused by affixed vascular adhesions during the lead extraction procedure.

Those of skill in the art will appreciate that the various illustrative logical blocks, modules, circuits, and algorithm steps described in connection with the embodiments disclosed herein may be implemented as electronic hardware, computer software, or combinations of both. Some of the embodiments and implementations are described above in terms of functional and/or logical block components (or modules) and various processing steps.

However, it should be appreciated that such block components (or modules) may be realized by any number of hardware, software, and/or firmware components configured to perform the specified functions. To clearly illustrate the interchangeability of hardware, various illustrative components, blocks, modules, and steps have been described above generally in terms of their functionality. Whether such functionality is implemented as hardware or software depends upon the application and design constraints imposed on the overall system.

Skilled artisans may implement the described functionality in varying ways for each application, but such implementation decisions should not be interpreted as causing a departure from the scope of the present invention. In addition, those skilled in the art will appreciate that the embodiments described herein are merely exemplary implementations.

In this document, relational terms such as first and second, and the like may be used solely to distinguish one entity or action from another entity or action without necessarily requiring or implying any actual such relationship or order between such entities or actions. Numerical ordinals such as “first,” “second,” “third,” etc. simply denote different singles of a plurality and do not imply any order or sequence unless specifically defined by the claim language. The sequence of the text in any of the claims does not imply that process steps must be performed in a temporal or logical order according to such sequence unless it is specifically defined by the language of the claim. When “or” is used herein, it is the logical or mathematical or, also called the “inclusive or.” Accordingly, A or B is true for the three cases: A is true, B is true, and A and B are true. In some cases, the exclusive “or” is constructed with “and;” for example, “one from the set A and B” is true for the two cases: A is true, and B is true.

Furthermore, depending on the context, words such as “connect” or “coupled to” used in describing a relationship between different elements do not imply that a direct physical connection must be made between these elements. For example, two elements may be connected to each other physically, electronically, logically, or in any other manner, through one or more additional elements.

While at least one exemplary embodiment has been presented in the foregoing detailed description of the invention, it should be appreciated that a vast number of variations exist. It should also be appreciated that the exemplary embodiment or exemplary embodiments are only examples and are not intended to limit the scope, applicability, or configuration of the invention in any way. Rather, the foregoing detailed description will provide those skilled in the art with a convenient road map for implementing an exemplary embodiment of the invention. It is understood that various changes may be made in the function and arrangement of elements described in an exemplary embodiment without departing from the scope of the invention as set forth in the appended claims.

What is claimed is:

1. An apparatus comprising a lead extraction assistance device for use with a lead locking device (LLD) in a lead extraction procedure, the apparatus comprising:
a lead locking device (LLD);
a lead locking assist device coupled to the LLD wherein the lead locking assist device comprises:
an assembly configured with a center shaft that has an inlet for entry of the lead and configured with an outlet for exit of the LLD wherein an assembly clamps both an entered lead and an exited LLD; and
a module disposed in the assembly and coupled to both the entered lead and the exited LLD, the module configured to apply about the center shaft, a first action by a rotational motor to twist the entered lead, and a second action by a hammering device to impact with hammering actions to the entered lead to assist to draw out the lead and counter any resistance caused by affixed vascular adhesions during the lead extraction procedure.

2. The apparatus of claim 1, wherein the lead extraction assistance device is a non-invasive device that is exterior to a body cavity in the lead extraction procedure; and the hammering device at least comprises a hammer drill.

3. The apparatus of claim 2, further comprising:
a spur gear which is fixed to the center shaft, wherein the center shaft is coupled on one end to the entered lead and another end to the exited LLD, and mates to another gear of the rotational motor to translate rotational motion to the center shaft and to the entered lead for application of a twist-action to twist the lead to assist in the attempt to draw out the lead and counter any resistance caused by the affixed vascular adhesions during the lead extraction procedure.

4. The apparatus of claim 3, further comprising:
a housing of the lead extraction assistance device configured for ergonomic use in an application of a pulling action after locking of the LLD to lead, to draw out the lead in combination with the application of the twist-action and the impact action to draw out the lead and counter any resistance caused by affixed vascular adhesions during the lead extraction procedure.

5. The apparatus of claim 4, further comprising:
a set of pins coupled to a vane assembly with a vane collar that is attached to the assembly that is responsive to rotations of the center shaft by the rotational motor that causes the set of pins to rotate along a vane shaft of the vane assembly that results in a push action to push the vane shaft in an opposite direction and to compress a spring attached to the assembly until a pin reaches an end of a vane shaft causing a release action of the compressed spring causing a hammering action that impacts the lead to draw out the lead and counter any resistance caused by affixed vascular adhesions during the lead extraction procedure.

6. The apparatus of claim 5, further comprising:
an alternate hammering device configured with a collar and shaft wherein the collar and shaft each has opposing sawtooth designs that rotate to mimic the hammering action that impacts the lead to assist to draw out the lead and counter any resistance caused by affixed vascular adhesions during the lead extraction procedure.

7. An apparatus comprising a lead extraction assistance device for use with a lead locking device (LLD) in a lead extraction procedure, the apparatus comprising:
a lead locking device (LLD);
a lead locking assist device coupled to the LLD wherein the lead locking assist device comprises:
an assembly configured with a center shaft that has an inlet for entry of the lead and configured with an outlet for exit of the LLD wherein an assembly clamps both an entered lead and an exited LLD; and
a module disposed in the assembly and coupled to both the entered lead and the exited LLD, the module configured to apply about the center shaft, a first action of a continuous vibration by a vibration source to at least a clamped entered lead and a second action of twisting to at least the clamped entered lead whilst the continuous vibration is applied to assist in attempting a drawing out of the lead that is resistant to extraction because of at least affixed vascular adhesions.

8. The apparatus of claim 7, wherein the lead extraction assistance device is a non-invasive device that is exterior to a body cavity in the lead extraction procedure.

9. The apparatus of claim 8, wherein the vibration source comprises an eccentric rotation mass (ERM) motor, a linear resonant actuator (LRA), or a piezoelectric actuator.

10. The apparatus of claim 9, wherein the vibration source is coupled to the center shaft via a collar and a needle bearing to enable the center shaft to rotate whilst the vibration source is kept static.

11. The apparatus of claim 10, further comprising:
a spur gear which is fixed to the center shaft, wherein the center shaft is coupled on one end to the entered lead and another end to the exited LLD, and mates to another gear of a rotational motor to translate rotational motion to the center shaft and to the entered lead for application of a twist-action to twist the lead to assist in the attempt to draw out the lead and counter any resistance caused by the affixed vascular adhesions during the lead extraction procedure.

12. The apparatus of claim 11, further comprising:
a housing of the lead extraction assistance device configured for ergonomic use in an application of a pulling action after locking of the LLD to lead, to draw out the lead in combination with the application of the twist-action and vibration action to draw out the lead and counter any resistance caused by affixed vascular adhesions during the lead extraction procedure.

13. An apparatus comprising a lead extraction assistance device for use with a lead locking device (LLD) in a lead extraction procedure, the apparatus comprising:
a lead locking device (LLD);
a lead locking assist device coupled to the LLD wherein the lead locking assist device comprises:
an assembly configured with a center shaft that has an inlet for entry of the lead and configured with an outlet for exit of the LLD wherein an assembly clamps both an entered lead and an exited LLD; and
a module disposed in the assembly and coupled to both the entered lead and the exited LLD, the module configured to apply about the center shaft, a first action by a rotational motor to twist at least a clamped entered lead, a second action by a hammering device to impact the clamped entered lead, and a third action by a vibration source to apply a continuous vibration to the clamped entered lead wherein the third action of the continuous vibration is applied during application of either the first action by the rotational motor of twisting the clamped entered lead or the second action by the hammering device of impacting the clamped entered lead to assist to draw out the lead and counter any resistance caused by affixed vascular adhesions during the lead extraction procedure.

14. The apparatus of claim 13, wherein the lead extraction assistance device is a non-invasive device that is exterior to a body cavity in the lead extraction procedure.

15. The apparatus of claim 14, further comprising:
a spur gear which is fixed to the center shaft that is coupled on either end to the entered lead and the exited LLD and mates to another gear of the rotational motor that translates rotational motion to the center shaft and to at least the entered lead to apply a twist-action to twist the lead to assist to draw out the lead and counter any resistance caused by affixed vascular adhesions during the lead extraction procedure.

16. The apparatus of claim 15, further comprising:
a housing of the lead extraction assistance device configured for ergonomic use in an application of a pulling action after locking of the LLD to lead, to draw out the lead in combination with the application of the twist-action, the impact action, and vibration action to draw out the lead and counter any resistance caused by affixed vascular adhesions during the lead extraction procedure.

17. The apparatus of claim 16, further comprising:
a set of pins coupled to a vane assembly with a vane collar that is attached to the assembly that is responsive to rotations of the center shaft by a motor that causes the set of pins to rotate along a vane shaft of the vane assembly that results in a push action to push the vane shaft in an opposite direction to compress a spring attached to the assembly until a pin of the set of pins reaches an end of the vane shaft causing a release action of the compressed spring causing a hammering action that impacts the lead to assist to draw out the lead and counter any resistance caused by affixed vascular adhesions during the lead extraction procedure.

18. The apparatus of claim 17, further comprising:
an alternate hammering device configured with a collar and shaft wherein the collar and shaft each has opposing sawtooth designs that rotate to mimic the hammering action that impacts the lead to assist to draw out the lead and counter any resistance caused by affixed vascular adhesions during the lead extraction procedure.

19. The apparatus of claim 18, wherein the vibration source comprises an eccentric rotation mass (ERM) motor, a linear resonant actuator (LRA), or a piezoelectric actuator.

20. The apparatus of claim 19, wherein the vibration source is coupled to the center shaft via a collar and a needle bearing to enable the center shaft to rotate whilst the vibration source is kept static.
